(19) United States
(12) Patent Application Publication
(10) Pub. No.: US 2004/0247707 A1
(43) Pub. Date: Dec. 9, 2004

(54) Pearl blue fast acting, long lasting topical pain relief cream

(76) Inventors: Charles Richard Le Roy II, Stanfield, AZ (US); Deborah Gay Le Roy, Stanfield, AZ (US)

Correspondence Address: Charles R. Le Roy II, P.O. Box 765, Stanfield, AZ, 85272, US

(21) Appl. No.: 10/750,519

(22) Filed: Dec. 31, 2003

Related U.S. Application Data
(60) Provisional application No. 60/437,902, filed on Jan. 6, 2003.

(51) Int. Cl. A61K 35/78; A61K 31/60; A61K 31/24

(52) U.S. Cl. 424/735; 424/744; 424/769; 514/165; 514/537

(57) ABSTRACT

Most pain relief products on the market, if they work at all are labeled “for temporary relief”. A person in pain for whatever reason desires three things. Since pain is not enjoyable, quick action is the first. For the same reason, long lasting is the second. The third consideration is as safe a product as possible. “Pearl Blue” performs all these needs quite well. The lidocaine is quick, begins relief in some degree in minutes. The other 6 ingredients penetrate at different rates so that as one diminishes the others in succession are working down the time chart to bring their own individual relief for up to 3 hours or longer depending on the sufferers own body chemistry. And the ingredients work together to aid each other in the whole result and have been time tested individually to be safe for generations.

### CROSS-REFERENCE TO RELATED APPLICATIONS

This application submitted in addition to Provisional Patent Application number 60/437,902 dated Jan. 6, 2003 and by the Title of Pearl Blue fast acting, long lasting topical pain relief cream. Pearl Blue, Works Like A Miracle is a Registered Trademark. Reg. number 2,781,627 Registered Nov. 11, 2003.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable,

### BACKGROUND OF THE INVENTION

In the century in which we live, not only have we inherited the various aches of age and those “normal pains” of injury, but a new list such as carpal tunnel syndrome, fibromyalgia and one of the most painful and sometimes long lasting medical conditions called “shingles”, which the prestigious “Family Physicians” claims 2 out of 10 adults who have had the childhood ailment chicken pox will acquire. We, a generation of “new” medical problems such as Gulf War Syndrome, not easy to diagnose yet, but with this great need for a genuine, honest working formula for topical use to relieve pain, the industry has fallen far short. It would seem that the major drug companies feel there is not enough profit here to put the money into development, and any consumer seeking true relief will quickly find that most over the counter pain relief creams may not work at all, and if they work at all are short lived. Many are so marked “for temporary relief” and these do not allow serious pain sufferers to even sleep through the night. After personal experience with severe pain and no over the counter remedy available from any source, and not wanting to take any long term potentially dangerous drugs, we knew this gap needed to be filled and have endeavored to do so. When pain occurs, the sufferer first wants quick action, one that truly works, and for chronic sufferers it should be long lasting. Even on the labels of these products you usually find the words “for temporary relief”. If those on the market work at all they are slow to act, and again if they work at all are not long lasting. Basically they just do not meet the needs of pain sufferers. Some noted in the field claim even minor pain will cost employers over $80 billion dollars this year alone, again stated, the profit margin does not seem to draw medical research in this direction. The need is great the supply is little.

### BRIEF SUMMARY OF THE INVENTION

As stated, the person with serious pain needs a serious solution. It must be fast acting and long lasting. The endeavor to do so is represented here in this way. There are pain receptors on the surface of the skin, and pain receptors below the surface in the sub-dermal tissue. Therefore one of the first remedies is to deal with both, and to do so it's pain relief must act quickly on the surface and super penetrate to carry the relief deep to relieve pain in the sub-dermal. First then, our invention “Pearl Blue” uses lidocaine HCL, as anesthetic, often applied to burns so in the first case, the lidocaine works almost immediately, but is short lived. Menthol and oil of wintergreen opens the pores of the skin to allow the other pain relief ingredients to penetrate while the lidocaine works. While this action is taking place, the very stable and penetrating trolamine salicylate is on it's way to the pain which is more slowly absorbed, for longer lasting relief. As these actions are taking place the aloe vera is also penetrating and adds to the penetrating power of the other pain relief ingredients. The sweet almond oil, a known pain reducer from Grandma's day, actually acts as a pain reducer and fills in the time gaps allowing, when all combined, to work within minutes and lasting for hours. We have achieved our goal.

### BRIEF DESCRIPTION OF THE SEVERAL VIEWS OF THE DRAWING

Not applicable.

### DETAILED DESCRIPTION OF THE INVENTION

### “Pearl Blue” Topical Pain Relief Cream

In our best to comply with USPTO requirements, as we understand them, the following should help. When we say “adequate quantity” we wish to point out our meaning. One of the ingredients of “Pearl Blue” is lidocaine HCL. Thus we say adequate, we refer to the percent of strength of lidocaine, which must be adequate to provide the pain relief, but the amount allowed by the FDA at any time may change, this more may be allowed. Our use is moderate according to FDA standards and we may increase or decrease the amount at any time, thus the patent request is not so dependent on the amount of each ingredient, but the combination of all, thus to allow latitude if we may improve our product and prevent any other party from merely increasing or decreasing any given amount and calling this new or improved. I have minimized the particular quantities of each ingredient. Indeed, increasing the amounts may aid specific injuries, and we are even now experimentally adding other ingredients to the “Base” or the beginning 7 ingredients. The “Base” should be considered an infringement of our application, or changing specific quantities of the “Base” ingredients. It is the combination then of these ingredients we wish to protect, for each ingredient used in “Pearl Blue” has its own value in the finished product, and the product works so well by the act of this combination. To properly prepare “Pearl Blue” the following ingredients are combined in a stainless steel vessel, and premixed with a glass or stainless steel rod to prevent any foreign ingredient from entering the mixture. Plastic should not be used in this process. Surgical gloves should also be worn, and sanitary procedures should be used throughout the mixing area. In the above stated vessel, an adequate amount of Lidocaine %, asprin %, aloe vera, tea tree oil, menthol, sweet almond oil and 2 teaspoons only per U.S. ½ gallon of oil of wintergreen, are hand blended slowly until color of mixture is nearly uniform. To the above ingredients is added a sufficient amount of any standard hand lotion formula to bring the total amount to one U.S. ½ gallon. At this time this total amount is added to a laboratory blender and mixed 20 to 30 minutes at a very slow speed. It is important to use minimum speed to prevent excess oxygen from invading the formula, which would shorten its shelf life. This final well blended mixture is then placed in sterile stock jars and sealed immediately, and is now ready for packaging and sale. As previously stated, current pain products cannot be labeled both fast acting and long lasting honestly. Our endeavor is to provide fast pain relief, long lasting pain relief and safe pain relief which we have succeeded in doing with “Pearl Blue”. The following is a breakdown of how this is accomplished and how the various ingredients accomplish their own tasks:

1. Lidocaine HCL, 0.4% per ½ gallon—an external analgesic, has a numbing effect on the nerves at the source of pain, this action is indeed extremely fast, and has been used for many years safely for serious burns because of this fact. However, this effect could not be called long lasting.

2. Trolamine Salicylate—asprin, ½ ounce per ½ gallon, a transdermal analgesic. A long lasting pain reliever said to have been given to the first to land on this continent by the Indians in the form of the bark of a tree that produces it. But, we had to wait until it was finally reduced to a very stable and penetrating form (trolamine salicylate). This starts to penetrate almost immediately—on it's way deeper pain or the place the pain originates from.

3. Aloe Vera, 2½ fluid ounces of gel per ½ gallon, an anti-inflammatory (pain reliever) from a southwestern cactus plant known for years for it's soothing properties. It is also a penetrant and helps to carry the other ingredients into the pained area in it's own time slot to further add to the long lasting effect of “Pearl Blue”.

4. Sweet Almond Oil, ¼ fluid ounce per ½ gallon, a non-penetrating oil that seals ingredients in and still allows the skin to breath and restore temporary elasticity, thereby helping to decrease irritation. Used in America by early physicians when heated for ear aches.

5. Menthol, 2 grams of crystal per ½ gallon, antibacterial properties. Causes a cooling sensation on the skin surface and opens the pores of the skin to enhance penetration of additional ingredients.

6. Oil of Wintergreen—Gaultheris procumbens, ⅙ fluid ounce per ½ gallon. Known as an excellent heat rub and slightly warms the pained area stimulating increased blood flow and also used as an aromatic to mask the musty smell of the tea tree oil which some find offensive. Also has mild antiseptic properties to maintain sterility of the cream remaining in the jar.

7. Tea Tree Oil—Melaleuca, ¼ fluid ounce per ½ gallon. Extract from the altemifolia tree in Australia. An antibacterial and antifungal agent. This is added to the formula to help sterilize and soothe the irritated area and upper dermal pain receptors. Thus reducing pain.

Thus all ingredients work together to bring immediate relief and due to their varying time of penetration continue for up to 3 hours and sometimes as long as 6 hours. Our tests on many types of pain show relief in minutes that truly lasts for hours with only 1 application, 2 needed in severe cases. Those products on the market, generally by our trials, did not work at all, and if they did, are short lived. Those with severe pain are not able to sleep without sedatives. This product allows many to sleep through the night as there is long enough pain relief for them to go to sleep where the body's own mechanisms go into effect, without narcotics. These ingredients combine to penetrate so well that after 20 minutes it can not be found. The pleasant wintergreen aroma fades to no aroma at all, and there is no oily residue and “Pearl Blue” will not stain clothes.

What we claim as our invention is:

1. When the seven (7) major ingredients of “Pearl Blue”, a topical pain relief cream or lotion, are properly combined, those ingredients being: 1. Lidocaine HCL, 2. Trolamine Salicylate—asprin, 3. Aloe Vera, 4. Sweet Almond Oil, 5. Menthol, 6. Oil of Wintergreen—Gaultheris procumbens, 7. Tea Tree Oil—Melaleuca, that they all work both individually and together in their respective categories, according to each items ability to penetrate to the pained area according to its own time of penetration to produce pain relief starting in minutes and lasting hours according to each individuals own body chemistry to be thus fast acting and long lasting. I have minimized the particular quantities of each ingredient, indeed, increasing the amounts may aid pain relief of specific injuries, and we are even now experimentally adding other ingredients, to the “Base” or the original 7 ingredients. The “Base” ingredients work well, although adding other ingredients to this “Base” may help, without the “Base” they, on their own, would not perform as well, so by adding other ingredients to the “Base” 7 ingredients, or varying their individual amounts should be considered an infringement of this patent process.
I. We believe the combination of the seven ingredients above listed as 1 thru 7 to be both unique and original and thus request a patent be granted to us to protect this highly successful formula.
II. We believe the 2 ingredients, Lidocaine HCL and Trolamne Salicylate, combined and used as a topical pain relief lotion or cream to be also unique and original, and request patent protection for his combination used as stated.
